(19) United States
(12) Patent Application Publication
(10) Pub. No.: US 2022/0370659 A1
(43) Pub. Date: Nov. 24, 2022

(54) REMOVABLY ATTACHABLE DISINFECTING LIGHT FOR A CONVEYOR BELT

(71) Applicant: Steribin, LLC, St. George, UT (US)

(72) Inventors: Wayne Provost, Ivins, UT (US); Jonathan M. Cole, St. George, UT (US); Jeffrey Mel Glauser, Santa Clara, UT (US)

(73) Assignee: Steribin, LLC, St. George, UT (US)

(21) Appl. No.: 17/745,688

(22) Filed: May 16, 2022

Related U.S. Application Data
(60) Provisional application No. 63/208,311, filed on Jun. 8, 2021.
(60) Provisional application No. 63/190,366, filed on May 19, 2021.

(51) Int. Cl. A61L 2/10; B65G 45/10

(52) U.S. Cl. CPC A61L 2/10; B65G 45/10; A61L 2202/11; A61L 2202/122; A61L 2202/121; A61L 2202/15

(57) ABSTRACT

The present disclosure relates to a removably attachable disinfecting device to disinfect items on a conveyor. The disinfecting device has a housing, an inlet, an outlet, and at least one disinfecting light positioned within the housing. The housing is five-sided and has an inlet cover and an outlet cover, a first lip on a first side, and a second lip on a second side, the lips configured to rest on an outer, stationary surface of a conveyor belt system. As an item passes down the conveyor belt, it enters the disinfecting device via the inlet where it is exposed to disinfecting light (e.g., UV light). The disinfected item proceeds to exit the disinfecting device on the conveyor belt via the outlet.

### CROSS-REFERENCE TO RELATED APPLICATIONS

This application claims priority to U.S. Provisional Application Ser. No. 63/190,366, filed on May 19, 2021, and further claims priority to U.S. Provisional Application Ser. No. 63/208,311, filed on Jun. 8, 2021, both of which are incorporated herein by reference.

### TECHNICAL FIELD

The present disclosure relates to a device to disinfect items. More particularly, the present disclosure relates to a device for disinfecting items on a conveyor.

### BACKGROUND

Infectious diseases commonly spread through the direct transfer of bacteria, viruses, or other microbes from contact with contaminated surfaces. Accordingly, disinfecting surfaces of items is important to maintaining health throughout the world. When surfaces are not disinfected to remove the viruses and bacteria thereon, people may become ill. Manufacturers and others have sought to disinfect items while they pass on conveyor belts. However, current disinfectant systems in the art require modifications to the conveyor system and are customized for the particular conveyor and goods thereon. This makes the installation expensive and burdensome. Additionally, it may require the conveyor to be shut down for installation, costing the company production and loss of money. As a result, companies who have conveyor systems do not want to make modifications to their line due to the high cost and cumbersome process.

Accordingly, there is a need for a disinfectant device for a conveyor belt that may be retrofitted to existing conveyor belts without requiring changes to the conveyor belt or its flow of products. The present disclosure seeks to solve these and other problems.

### SUMMARY OF EXAMPLE EMBODIMENTS

In some embodiments, a removable attachable disinfecting device for conveyors comprises a housing, an inlet, an outlet, and at least one disinfecting light positioned within the housing. In some embodiments, the housing is five-sided and comprises an inlet cover and an outlet cover, a first lip on a first side and a second lip on a second side, the lips configured to rest on an outer, stationary surface of a conveyor belt system. In some embodiments, the removable attachable disinfecting device for conveyors may further comprise one or more circulation fans. In some embodiments, the housing comprises handles.

In one method of use, a removable attachable disinfecting device for conveyors is positioned to rest atop a conveyor belt system. As an item passes down the conveyor belt, it enters the disinfecting device via the inlet where it is exposed to disinfecting light (e.g., UV light). The disinfected item proceeds to exit the disinfecting device on the conveyor belt via the outlet.

### BRIEF DESCRIPTION OF THE DRAWINGS

FIG. 1 illustrates a top, right side perspective view of a removable attachable disinfecting device for conveyors;

FIG. 2 illustrates a side longitudinal cross-section of a removable attachable disinfecting device for conveyors;

FIG. 3 illustrates a left side elevation view of a removable attachable disinfecting device for conveyors;

FIG. 4 illustrates a top plan view of a removable attachable disinfecting device for conveyors; and

FIG. 5 illustrates a bottom plan view of a removable attachable disinfecting device for conveyors.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.). While ultraviolet (UV) light is used as an example throughout, it will be appreciated that any light, or electromagnetic wavelength of light, capable of destroying or inhibiting the growth of microorganisms is contemplated herein as a “disinfecting light.”

As previously discussed, there is a need for a disinfectant device for a conveyor belt that may be retrofitted to existing conveyor belts without requiring changes to the conveyor belt or its flow of products. The removably attachable disinfecting device for conveyors disclosed herein seeks to solve these and other problems.

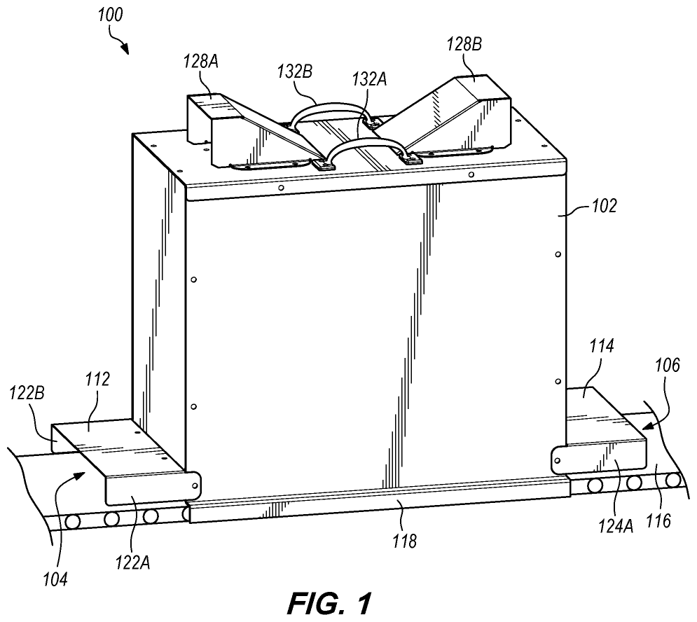

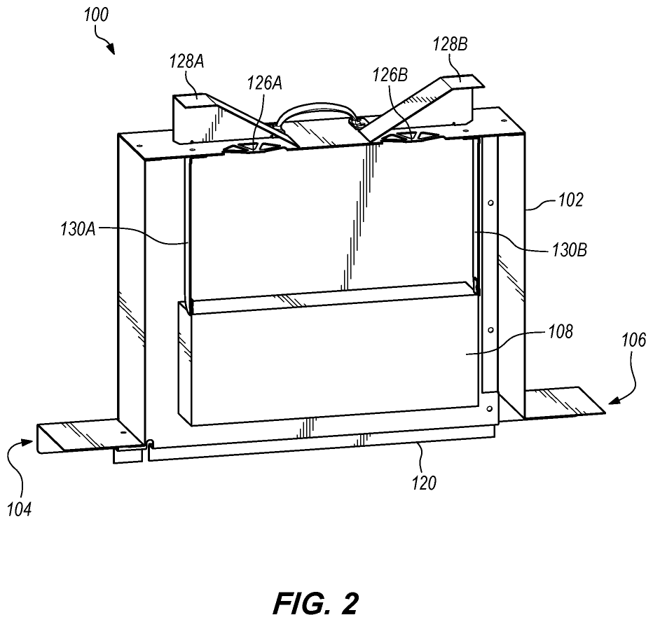

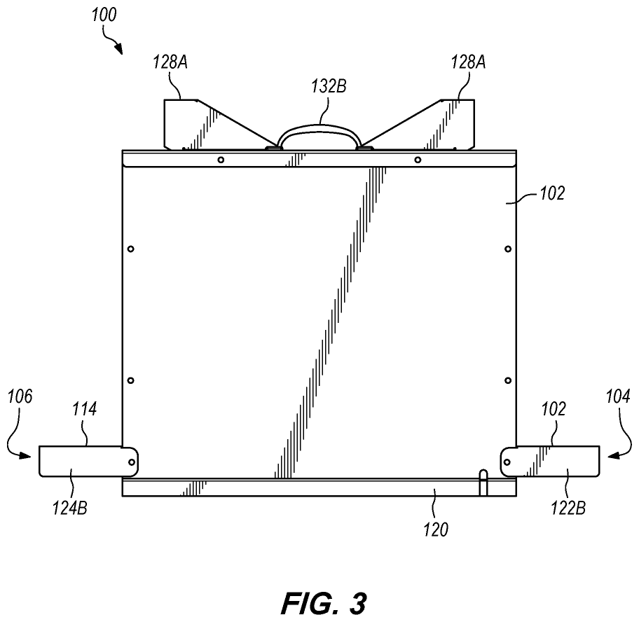

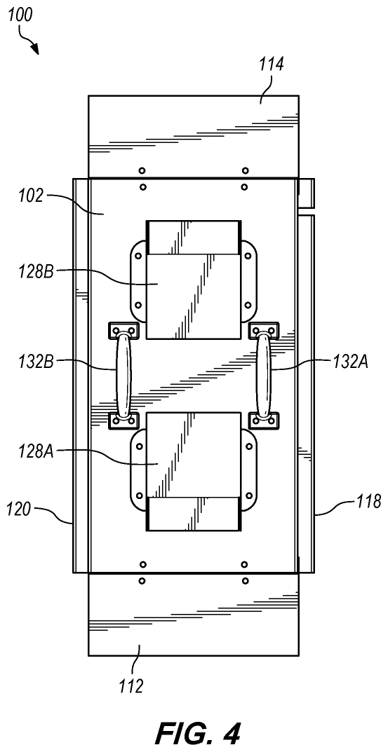

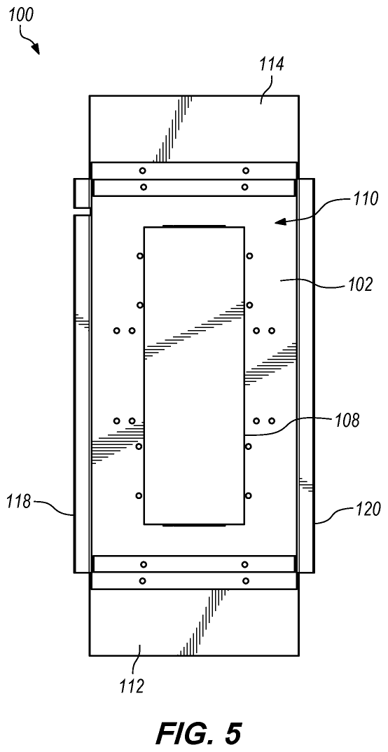

In some embodiments, as shown in FIGS. 1-5, a removable attachable disinfecting device 100 for conveyors comprises a housing 102, an inlet 104, an outlet 106, and at least one disinfecting light 108 positioned within the housing 102. It will be appreciated that the disinfecting light 108 may comprise a light housing 109 or may simply be bulbs or other light sources. Additionally, there may be one or a plurality of disinfecting lights 108 within the housing 102 and/or light housing 109. In some embodiments, the housing 102 is five-sided and generally comprises an open bottom 110 for resting over a conveyor belt 116. The removable attachable disinfecting device 100 may comprise an inlet cover 112 and an outlet cover 114 to help reduce the amount of light that may escape the housing 102 and to help ensure a maximum height of items on the conveyor belt 116. The housing 102 further comprises a first lip 118 on a first side and a second lip 120 on a second side, the lips 118, 120 configured to rest on an outer, stationary surface of a conveyor belt system 116. For example, the housing may be placed so that the first and second lips 118, 120 engage a first stationary side and a second stationary side of a conveyor belt 116, respectively. This ensures that the housing 102 does not come into contact with the conveyor belt 116 so that the housing 102 remains stationary despite the conveyor 116 passing thereunder. Clamps, bolts, or other securing mechanisms may also be used to secure the lips 118, 120 to the stationary sides, if desired, although not required.

The inlet cover 112 is positioned above the conveyor belt 116 to aid in blocking light from escaping the housing 102 and to ensure a maximum height of items is maintained. For example, if nuts or berries are passing down the conveyor 116 and become piled up, the inlet cover distributes the pile so that items enter the housing 102 at a predetermined height. The inlet cover 112 may comprise sidewalls 122A-B to aid in keeping light from escaping and to ensure items do not fall off the conveyor 116 when entering the inlet 104. Likewise, the outlet cover 114 may comprise sidewalls 124A-B to aid in keeping light from escaping the housing 102.

In some embodiments, the removable attachable disinfecting device 100 for conveyors may further comprise one or more circulation fans 126A-B, which may be covered by fan shrouds 128A-B to prevent unwanted items from contacting the fans 126A-B. The circulation fans 126A-B help to dissipate heat within the housing 102. The disinfecting light 108 is positioned within the housing 102 so that light shines on the conveyor belt 116 and the items passing thereon. In some embodiments, the distance between the conveyor belt 116 and the disinfecting light 108 may be adjustable, such as by using adjustable brackets 130A-B, or other suitable devices. For example, it may be desirable for some items to have less exposure to UV light, in which case the disinfecting light 108 may be raised farther from the conveyor 116. In one embodiment, the adjustable brackets 130A-B comprise groove that mates with a tongue of the disinfecting light, allowing the disinfecting light to be slidable therein. Once the desired position is achieved, a user may secure the disinfecting light to the brackets 130A-B using bolts, cam locks, or other means. In some embodiments, different sized brackets 130A-B may be used to vary the height. Additionally, the disinfecting light 108 can be configured widthwise or lengthwise within the housing 102 in order to increase or decrease disinfection exposure/time.

In some embodiments, the housing 102 comprises handles 132A-B coupled thereto, allowing a user to easily lift and maneuver the removable attachable disinfecting device 100. Accordingly, in one method of use, a user may lift and place a removable attachable disinfecting device 100 atop a conveyor 116 with a first lip 118 on a first side and a second lip 120 on a second side. Once positioned, items may enter the removable attachable disinfecting device 100 via the inlet 104, be exposed to the disinfecting light 108 while traveling within the housing 102, and then exit the housing at outlet 106. Because the removable attachable disinfecting device 100 is easily placed atop a conveyor 116, no modifications to the conveyor 116 are required. This ensures little downtime for installation of the removable attachable disinfecting device 100. Further, if the need for disinfection is no longer required, removing the removable attachable disinfecting device 100 is easily achieved by simply lifting the device off of the conveyor 116.

Additionally, the removable attachable disinfecting device 100 may comprise legs that allow the housing 102 to be suspended/hover above the conveyor 116. The legs may be adjustable (e.g., telescopic) so as to be used on conveyors 116 of various heights and/or to vary exposure time of items on the conveyor 116 to the disinfecting light 108.

Further, although generally referred to herein as a “disinfecting device,” it is understood that a disinfecting device of the present disclosure may disinfect, sterilize, sanitize, or otherwise treat and clean the surface of a contaminated item to achieve a lessened state or condition of contamination. Housing 102 may include an exterior material having a first property or function, and an interior material having a second property or function that is different than the first property or function. For example, in at least one embodiment, the housing 102 may include an exterior material that is structurally rigid and opaque, and an interior material that is reflective. In some embodiments, the interior material of the housing 102 may include a coating applied to an inner surface of the exterior material of the housing 102.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A removably attachable disinfectant device for a conveyor, comprising:
a housing having five sides;
an inlet;
an outlet;
a first lip for resting on a first side of the conveyor;
a second lip for resting on a second side of the conveyor; and
at least one disinfecting light positioned within the housing.

2. The removably attachable disinfectant device of claim 1, further comprising an inlet cover and an outlet cover.

3. The removably attachable disinfectant device of claim 1, further comprising at least one circulation fan and a fan shroud.

4. The removably attachable disinfectant device of claim 1, further comprising at least one handle coupled to the housing.

5. The removably attachable disinfectant device of claim 1, further comprising adjustable brackets configured to couple the disinfecting light to the housing.

6. A removably attachable disinfectant device for a conveyor, comprising:
a housing having five sides;
an inlet on a first side, the inlet comprising an inlet over extending substantially perpendicular to the housing;
an outlet on a second side, the outlet comprising an outlet cover substantially perpendicular to the housing;
a first lip for resting on a first, stationary side of the conveyor;
a second lip for resting on a second, stationary side of the conveyor;
at least one disinfecting light positioned within the housing; and
at least one circulation fan configured to dissipate heat from an interior of the housing.

7. The removably attachable disinfectant device of claim 6, further comprising adjustable brackets configured to couple the disinfecting light to the housing.

8. The removably attachable disinfectant device of claim 6, further comprising a plurality of handles coupled to the housing.

9. A method of disinfecting items on a conveyor belt using a removably attachable disinfectant device for conveyors, the method comprising:
placing the removably attachable disinfectant device over a conveyor belt, the conveyor belt interposed between a first lip and a second lip of a housing, the removably attachable disinfectant device comprising an inlet, at least one disinfecting light positioned within the housing, and an outlet;
feeding items through the inlet, under the disinfecting light within the housing, and out of the outlet via the conveyor belt.

10. The method of claim 9, wherein the removably attachable disinfectant device further comprises an inlet cover and an outlet cover.

11. The removably attachable disinfectant device of claim 9, wherein the removably attachable disinfectant device further comprises at least one circulation fan and a fan shroud.

12. The removably attachable disinfectant device of claim 1, wherein the removably attachable disinfectant device further comprises at least one handle coupled to the housing.

13. The removably attachable disinfectant device of claim 1, wherein the removably attachable disinfectant device further comprises adjustable brackets configured to couple the disinfecting light to the housing.
